(19) United States
(12) Patent Application Publication
(10) Pub. No.: US 2021/0187211 A1
(43) Pub. Date: Jun. 24, 2021

(54) NEBULIZERS, NEBULIZER CARTRIDGES AND USES THEREOF

(71) Applicant: OMEGA LIFE SCIENCE LTD., Migdal Haemeq (IL)

(72) Inventor: Miron HAZANI, Haifa (IL)

(21) Appl. No.: 17/053,178

(22) PCT Filed: May 5, 2019
(86) PCT No.: PCT/IL2019/050497

§ 371 Date: Nov. 5, 2020

Related U.S. Application Data
(60) Provisional application No. 62/668,263, filed on May 8, 2018.
(60) Provisional application No. 62/689,844, filed on Jun. 26, 2018.

(51) Int. Cl. A61M 11/02; A61M 15/00; A24F 40/05; A24F 40/10; A24F 40/44; A24F 40/48; A24F 40/42

(52) U.S. Cl. CPC A61M 11/02; A61M 15/0021; A24F 40/05; A24F 40/42; A24F 40/44; A24F 40/48; A24F 40/10

(57) ABSTRACT

The present disclosure generally relates to the field of nebulizers for aerosol generation and methods of using same for treating diseases and disorders.

### TECHNICAL FIELD

The present disclosure generally relates to the field of nebulizers for aerosol generation and methods of using same for treating diseases and disorders.

### BACKGROUND

Nebulizers are commonly used for delivering aerosol medication to patients via the respiratory system. Desirably, for efficient delivery of medication, the aerosol should include droplets having droplet diameter sufficiently small so as to reach the lungs of the patient without being obstructed by objects or organs (such as, the inner surface of the nozzle in the nebulizer and the mouth cavity perimeters) and large enough so as to remain in the lungs during exhalation.

The main techniques for producing aerosol in nebulizers include vibrating Mesh technology, jet nebulizers and ultrasonic wave nebulizers. Common to these techniques is the challenge to deliver large volume of medication to the patient while keeping the diameter of the droplets within desired limits.

WO 2016/059630 to the inventor of the present invention discloses a nebulizer comprising a porous medium configured to produce aerosols, a displaceable wetting mechanism configured to spread a liquid over the porous medium thereby to wet the porous medium and a gas channel configured to introduce pressure gradient to the porous medium.

There is still need for improved wetting mechanisms for better spreading of pharmaceutical liquids in porous media for more efficient production of aerosols.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided herein devices and systems for generating aerosols for delivery of biologically active materials, such as respiratory tract medications and/or nicotine. The delivery is taking place using pressurized air, which flows through at least one porous medium in the nebulizer cartridge, thereby generating aerosol, which exits the nebulizer through a mouthpiece to the lungs of a user in need for an aerosol delivery. The generation of aerosol requires the wetting the porous medium with a composition of the active material(s). According to some embodiments, the nebulizer comprises a nebulizer cartridge, which comprises a combination of a liquid absorbing element(s) and a stationary liquid absorbing element(s), which enable efficient wetting of the porous medium. Basically, the stationary liquid absorbing element(s) extends from a liquid (e.g. a drug solution) containing reservoir to a track, on which the mobile liquid absorbing element(s) is moved. As a result, the stationary liquid absorbing element(s) absorbs the liquid from the reservoir, the mobile liquid absorbing element(s) absorbs the liquid from the stationary liquid absorbing element(s) upon their contact. Thereafter, the liquid-absorbed mobile liquid absorbing element(s) is moved using a motorized conveyer on the track, thus wetting the surface of the at least one porous medium and feeds it with the liquid. This course may repeat several times until sufficient wetting of the porous medium is achieved. Upon sufficient wetting of the porous medium, pressurized air may be applied therethrough, and aerosol is formed.

According to some embodiments, there is provided a nebulizer cartridge comprising,
at least one porous medium having an proximal surface, the at least one porous medium extending between a first position and a second position;
at least one reservoir configured to contain a liquid;
at least one mobile liquid absorbing element;
at least one stationary liquid absorbing element being in contact with the at least one reservoir;
at least one conveyer connected to the at least one mobile liquid absorbing element, and configured to be actuated by a motor; and
a track operably linked to the at least one conveyer,
wherein said at least one conveyer and said at least one mobile liquid absorbing element connected thereto are configured to move along the track; and wherein the at least one mobile liquid absorbing element is configured to be in contact with the at least one stationary liquid absorbing element, and upon movement along the track it is further configured to be in contact with the at least one porous medium.

According to some embodiments, the at least one reservoir contains the liquid, wherein said at least one stationary liquid absorbing element is in contact with the liquid contained in the reservoir.

According to some embodiments, each of the at least one stationary liquid absorbing element and the at least one mobile liquid absorbing element separately, is configured to absorb liquid in an amount which is at least 150% of its respective weight.

According to some embodiments, each of the at least one stationary liquid absorbing element and the at least one mobile liquid absorbing element comprises cloth, wool, felt, sponge, foam, cellulose, yarn, microfiber or a combination thereof.

According to some embodiments, the track extends along the first position and the second position of the at least one porous medium.

According to some embodiments, the nebulizer cartridge further comprises a mouthpiece, such that the proximal surface of the at least one porous medium is facing the mouthpiece.

According to some embodiments, the at least one porous medium is having an distal surface, opposing the proximal surface, and the nebulizer cartridge further comprises a pressurized air inlet, such that the distal surface of the at least one porous medium is facing the pressurized air inlet.

According to some embodiments, the at least one stationary liquid absorbing element contains a portion of the liquid, absorbed therein, and wherein the at least one mobile liquid absorbing element is in contact with the at least one stationary liquid absorbing element, thereby absorbing liquid therefrom.

According to some embodiments, the at least one mobile liquid absorbing element is in contact with the at least one porous medium, thereby wetting the proximal surface thereof.

According to some embodiments, the wetting comprises spreading.

According to some embodiments, the liquid comprises an aqueous solution or an aqueous suspension of a pharmaceutical composition.

According to some embodiments, the nebulizer cartridge comprises a plurality of porous media; a plurality of reservoirs, each containing a liquid; a plurality of mobile liquid absorbing elements; a plurality of stationary liquid absorbing elements; and a plurality of conveyers, each configured to be actuated by a respective motor.

According to some embodiments, each of said plurality of reservoirs contains a different liquid.

According to some embodiments, there is provided a nebulizer comprising:
the nebulizer cartridge disclosed herein, wherein the least one conveyer comprises a rack and pinion mechanism; and
a control unit;
wherein said control unit comprises a conveyer motor having a gear unit and a pressurized air source; wherein said nebulizer cartridge is configured to be mounted on the control unit, such that upon mounting, at least one cogwheel of the gear operates the rack and pinion mechanism, and the at least one conveyer is actuated by the conveyer motor.

According to some embodiments, the conveyer motor is configured to be actuated by a user.

According to some embodiments, the control unit comprises a computing unit configured to operate the conveyer motor.

According to some embodiments, the computing unit is controlled by a user.

According to some embodiments, the pressurized air source comprises an air pump.

According to some embodiments, the control unit comprises a pump motor, configured to operate the air pump.

According to some embodiments, the control unit comprises an electric power source, configured to power the computing unit, the pump motor and the conveyer motor.

According to some embodiments, the nebulizer cartridge further comprises a pressurized air inlet, configured to enable transfer of pressurized air from the pressurized air source to the nebulizer cartridge.

According to some embodiments, the nebulizer cartridge further comprises a mouthpiece, such that upon application of the pressurized air source, pressurized air flows therefrom, through the at least one porous medium, thereby producing aerosol, which flows out the nebulizer cartridge through the mouthpiece.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a nebulizer cartridge, according to some embodiments;

FIG. 2 schematically illustrates a nebulizer cartridge, according to some embodiments;

FIG. 3 schematically illustrates a nebulizer cartridge, according to some embodiments;

FIG. 4 schematically illustrates a nebulizer cartridge, according to some embodiments;

FIGS. 5A and 5B schematically illustrate a perspective sectional view of nebulizer, according to some embodiments.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a nebulizer cartridge comprising at least one porous medium; at least one reservoir configured to contain a liquid; at least one mobile liquid absorbing element; at least one stationary liquid absorbing element; and at least one conveyer configured to be actuated by a motor; wherein said at least one mobile liquid absorbing element is movable by the conveyer on a track; wherein said at least one stationary liquid absorbing element extends from the at least one reservoir to the track; and is in contact with the liquid, when contained in the reservoir, such that upon moving the at least one mobile liquid absorbing element on the track, the at least one mobile liquid absorbing element is at least temporarily in contact with the at least one stationary liquid absorbing element and at least temporarily in contact with the at least one porous medium.

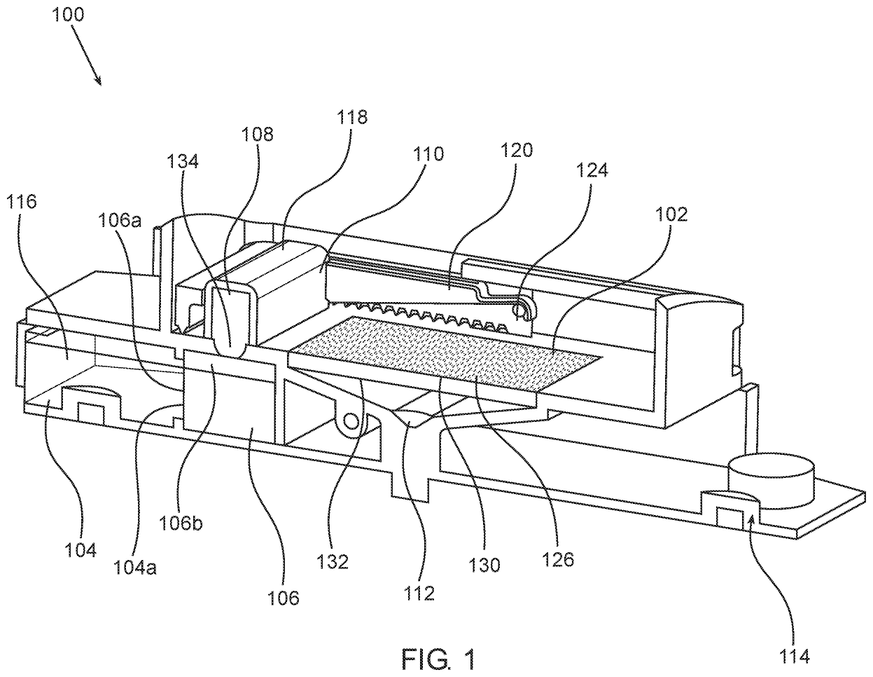

Reference is now made to FIGS. 1-4, which schematically illustrate a nebulizer cartridge. FIG. 1 schematically illustrate a nebulizer cartridge 100 comprising a porous medium 102, reservoir 104, a stationary liquid absorbing element 106, a mobile liquid absorbing element 108 at position 134, a conveyer 110, a pressurized air inlet 112 and a snap-fit 114, according to some embodiments.

According to some embodiments, the at least one reservoir contains a liquid. According to some embodiments, the at least one stationary liquid absorbing element is having a proximal surface facing the at least one reservoir and is in contact with the liquid contained in the reservoir, thereby absorbed with a first amount of the liquid. According to some embodiments, the at least one stationary liquid absorbing element is having an upper surface facing the at least one mobile liquid absorbing element at position 134. At this configuration, the mobile liquid absorbing element absorbs from the at least one stationary liquid absorbing element a portion of the first amount of the liquid.

According to some embodiments, reservoir 104 is a container for holding liquid. According to some embodiments, reservoir 104 contains a first amount of aqueous pharmaceutical composition 116. According to some embodiments, reservoir 104 is having a distal surface 104a facing a first surface 106a of stationary liquid absorbing element 106, and being in contact with aqueous pharmaceutical composition 116.

According to some embodiments, stationary liquid absorbing element 106 includes a first portion of the first amount of aqueous pharmaceutical composition 116 absorbed therein.

According to some embodiments, stationary liquid absorbing element 106 is a sponge. According to some embodiments, stationary liquid absorbing element 106 is a hydrophilic sponge.

According to some embodiments, mobile liquid absorbing element 108 is a sponge. According to some embodiments, mobile liquid absorbing element 108 is a hydrophilic sponge.

It is to be understood that a hydrophilic sponge has high tendency to absorb aqueous solutions.

The terms “liquid absorbing material”, “liquid absorbing element” and “liquid absorbent material” as used herein are interchangeable and refer to any material, or element comprising a material that is capable of incorporating, taking in, drawing in or soaking liquids, and upon applying physical pressure thereto or being in contact with another material, release a portion or the entire amount/volume of the absorbed liquid.

According to some embodiments, the at least one stationary liquid absorbing element is configured to absorb water in an amount which is at least 100% of its weight. According to some embodiments, the at least one stationary liquid absorbing element is configured to absorb water in an amount which is at least 150% of its weight. According to some embodiments, the at least one stationary liquid absorbing element is configured to absorb water in an amount which is at least 200% of its weight.

According to some embodiments, the at least one mobile liquid absorbing element is configured to absorb water in an amount which is at least 100% of its weight. According to some embodiments, the at least one mobile liquid absorbing element is configured to absorb water in an amount which is at least 150% of its weight. According to some embodiments, the at least one mobile liquid absorbing element is configured to absorb water in an amount which is at least 200% of its weight.

According to some embodiments, the at least one stationary liquid absorbing element comprises cloth, wool, felt, sponge, foam, cellulose, yarn, microfiber or a combination thereof. Each possibility represents a separate embodiment.

According to some embodiments, the at least one stationary liquid absorbing element comprises a sponge. According to some embodiments, the at least one stationary liquid absorbing element comprises a foam. According to some embodiments, the sponge is an open cell sponge. According to some embodiments, the sponge is a closed cell sponge. According to some embodiments, the at least one stationary liquid absorbing element comprises fabric. Specifically, fibrous and/or woven fabric, such as a wick, is a hydrophilic and water absorbing material, which may be used as the stationary liquid absorbing element(s), according to some embodiments.

According to some embodiments, the at least one mobile liquid absorbing element comprises cloth, wool, felt, sponge, foam, cellulose, yarn, microfiber or a combination thereof. Each possibility represents a separate embodiment.

According to some embodiments, the at least one mobile liquid absorbing element is similar in texture to the at least one stationary liquid absorbing element, described herein. For example, the at least one mobile liquid absorbing element comprises a sponge, a foam (closed cell sponge or open cell sponge), fabric and the like.

Without wishing to be bound by any theory or mechanism of action, when the liquid is a water-based pharmaceutical composition, hydrophilic mobile- and/or stationary liquid absorbing element(s) are preferred. In this situation, the aqueous composition in the reservoir(s) is efficiently absorbed in the stationary liquid absorbing element(s); and therefrom it absorbs in the mobile liquid absorbing element(s) to create equilibrium. Consequently, the absorbed mobile liquid absorbing element(s) delivers the aqueous composition to the at least one porous medium to produce the desired aerosol. In addition, when the at least one stationary liquid absorbing element comprises a hydrophilic sponge, as it comes in contact with the aqueous pharmaceutical composition in the reservoir, capillary action within and among the pores of the sponge lead to absorption of the aqueous pharmaceutical composition therein. The same capillary action results with the absorption of the aqueous pharmaceutical composition by the at least one mobile liquid absorbing element.

According to some embodiments, the at least one mobile liquid absorbing element is hydrophilic. According to some embodiments, the at least one mobile liquid absorbing element is a hydrophilic sponge. Likewise, according to some embodiments, the at least one stationary liquid absorbing element is hydrophilic, for example, a hydrophilic sponge.

The term “sponge” as used herein refers to any porous, wettable, cellular and/or foam-like type of material having a texture, which includes a plurality of open and/or closed pores.

The term “hydrophilic” material, as used herein, refers to any material which has a high affinity to water and/or that water has high affinity thereto. Preferably, hydrophilic materials according to the current disclosure have high capability to absorb water and aqueous solutions.

According to some embodiments, the at least one mobile liquid absorbing element and the at least one stationary liquid absorbing element are composed of the same material.

Referring again to FIG. 1, this figure illustrates a configuration where stationary liquid absorbing element 106 is in contact with mobile liquid absorbing element 108 which is in position 134 (hereinafter, “Configuration A”). As stationary liquid absorbing element 106 is in a fixed position and is in contact with the liquid contained in reservoir 104, it absorbs a portion of aqueous pharmaceutical composition 116 therefrom. Thus, stationary liquid absorbing element 106 is being absorbed with a portion of aqueous pharmaceutical composition 116. Furthermore, when mobile liquid absorbing element 108 is in position 134, as illustrated in FIG. 1, mobile liquid absorbing element 108 absorbs a portion of aqueous pharmaceutical composition 116 absorbed in stationary liquid absorbing element 106.

According to some embodiments, aqueous pharmaceutical composition 116 comprises a therapeutically effective amount of medication for treating one or more medical conditions, which affect the respiratory system.

According to some embodiments, the liquid comprises an aqueous solution or an aqueous suspension. According to some embodiments, the liquid comprises an aqueous solution.

According to some embodiments, the liquid comprises at least one biologically active material having an effect on the respiratory system. According to some embodiments, the liquid comprises a medication intended to be delivered to the lungs. According to some embodiments, the liquid comprises nicotine. According to some embodiments, the liquid comprises a composition comprising nicotine. According to some embodiments, the liquid comprises an aqueous composition comprising nicotine.

According to some embodiments, the liquid comprises a pharmaceutical composition. According to some embodiments, the pharmaceutical composition is for treating a disease via inhalation.

According to some embodiments, the pharmaceutical composition comprises one or more pharmaceutically active agents. According to some embodiments, the one or more pharmaceutically active agents are suitable or may be adjusted for inhalation. According to some embodiments, the one or more pharmaceutically active agents are directed for treatment of a medical condition through inhalation.

As used herein, a “pharmaceutical composition” refers to a preparation of a composition comprising one or more pharmaceutically active agents, suitable for administration to a patient via the respiratory system.

According to some embodiments, the pharmaceutical composition further comprises at least one pharmaceutical acceptable carrier. In other embodiments, the pharmaceutical composition may further comprise one or more stabilizers.

According to some embodiments, the nebulizer provides an aerosol containing a therapeutically effective amount of the pharmaceutical composition. As used herein, the term “therapeutically effective amount” refers to a pharmaceutically acceptable amount of a pharmaceutical composition which prevents or ameliorates at least partially, the symptoms signs of a particular disease, for example infectious or malignant disease, in a living organism to whom it is administered over some period of time.

The term “pharmaceutically acceptable” as used herein means approved by a regulatory agency of the Federal or a state government or listed in the U.S. Pharmacopeia or other generally recognized pharmacopeia for use in animals and, more particularly, in humans.

The pharmaceutical compositions of the invention may be prepared in any manner well known in the pharmaceutical art.

Useful pharmaceutically acceptable carriers are well known in the art, and include, for example, lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. Other pharmaceutical carriers can be sterile liquids, such as water, alcohols (e.g., ethanol) and lipid carriers such as oils (including those of petroleum, animal, vegetable or synthetic origin, such as peanut oil, soybean oil, mineral oil, sesame oil and the like), phospholipids (e.g. lecithin), polyethylene glycols, glycerine, propylene glycol or other synthetic solvents. Each possibility represents as separate embodiment of the present invention.

Pharmaceutical acceptable diluents include, but are not limited to, sterile water, phosphate saline, buffered saline, aqueous dextrose and glycerol solutions, and the like. Each possibility is a separate embodiment of the invention.

According to some embodiments, the at least one therapeutic agent is selected from the group consisting of a hormone, a steroid, anti-inflammatory agent, antibacterial agent, anti-neoplastic agent, pain relief agent, narcotics, anti-angiogenic agent, siRNA, immuno-therapy related agent, growth-inhibitory agent, apoptotic agent, cytotoxic agent and chemotherapeutic agent. Each possibility is a separate embodiment of the invention.

According to some embodiments, the pharmaceutical composition comprises albuterol, also known as, salbutamol and Ventolin®.

According to some embodiments, the medical condition is a pulmonary disease. According to some embodiments, the pulmonary disease is bronchospasm, asthma and chronic obstructive pulmonary disease among others. According to some embodiments, the asthma is allergen asthma or exercise-induced asthma.

According to some embodiments, the medical condition is a lung disease affecting the air ways, the alveoli or the interstitium, such as, asthma, chronic obstructive pulmonary disease, chronic bronchitis, emphysema, acute bronchitis, cystic fibrosis, pneumonia, tuberculosis, fragile connections between alveoli, pulmonary edema, lung cancer in its many forms, acute respiratory distress syndrome, pneumoconiosis, interstitial lung disease among others.

According to some embodiments, the pharmaceutical composition comprises a therapeutically effective amount of medication for treating one or more of the medical conditions stated herein.

In some embodiments the medical condition is a metabolic disease, such as, diabetes mellitus (diabetes) Type 1, Type 2 and gestational diabetes, and the at least one pharmaceutical composition comprises a therapeutically effective amount of inhalable insulin.

According to some embodiments, while the cartridge is disconnected (i.e. it is not connected to a nebulizer control unit, as depicted in FIG. 1) liquid absorbing element 108 is in position 134. According to some embodiments, while liquid absorbing element 108 is in position 134 the amount of aqueous pharmaceutical composition 116 in reservoir 104 is remained substantially constant.

Specifically, according to some embodiments, stationary liquid absorbing element 106 comprises a first surface 106a, and second surface 106b. According to some embodiments, first surface 106a is facing reservoir 104. According to some embodiments, first surface 106a is protruding into reservoir 104. According to some embodiments, stationary liquid absorbing element 106 is absorbing a portion of aqueous pharmaceutical composition 116 from reservoir 104 through first surface 106a. Thus, according to some embodiments, a portion of aqueous pharmaceutical composition 116 is absorbed into stationary liquid absorbing element 106. Thus, according to some embodiments, the amount of aqueous pharmaceutical composition 116 in reservoir 104 and the amount of aqueous pharmaceutical composition 116 in stationary liquid absorbing element 106 is in equilibrium. According to some embodiments, the amount of aqueous pharmaceutical composition 116 in reservoir 104 and the amount of aqueous pharmaceutical composition 116 in stationary liquid absorbing element 106 is in equilibrium, such that the amount of amount of aqueous pharmaceutical composition 116 in each of reservoir 104 and liquid absorbing element 106 is substantially constant, when nebulizer cartridge 100 is in Configuration A.

According to some embodiments, second surface 106b is facing mobile liquid absorbing element 108 where mobile liquid absorbing element 108 is nested within conveyer 110.

The term “surface”, as used herein, refers generally to any interface separating two media and/or phases. It intends to refer to a generalization of a plane which needs not be flat, i.e. the curvature of a surface is not necessarily zero.

Each one of first surface 106a and second surface 106b may be substantially flat, or curved, according to some embodiments. FIG. 1 refers to the former option, while, due to an amorphous shape, which sponges tend to have while soaked and/or squeezed, the latter option is contemplated. According to some embodiments, first surface 106a and second surface 106b may be a continuation of one another. For, example according to some embodiments, the surface of stationary liquid absorbing element 106 may be round, such that first surface 106a and second surface 106b partially overlaps at the convex surface of liquid absorbing element 106.

According to some embodiments, conveyer 110 comprises a retaining unit 118 and track 120. According to some embodiments, retaining unit 118 and track 120 are physically connected to each other.

According to some embodiments, conveyer 110 is retaining, encompassing, housing or nesting mobile liquid absorbing element 108. According to some embodiments, conveyer 110 comprises a retaining unit, configured to retain therein mobile liquid absorbing element 108, such that conveyer 110 and the at least one mobile liquid absorbing element 108 are moving together, as one unit. According to some embodiments, conveyer 110 comprises at least one rack-like element and at least one cogwheel. According to some embodiments, the at least one rack-like element is located along track 120. According to some embodiments, each of the at least one rack-like element and the at least one cogwheel comprises serrated teeth. According to some embodiments, the at least one cogwheel comprises an external cogwheel having serrated teeth. According to some embodiments, the serrated teeth of the at least one rack-like element are interlocking with the serrated teeth of the external cogwheel, such that upon rotating said interlocking external cogwheel, its teeth are rotating a radial direction, and pushing the interlocked teeth of the at least one rack-like element, such that the at least one rack-like element is moved at a tangential direction in a rack and pinion mechanism. According to some embodiments, rotating said external cogwheel in the opposite direction entails moving the at least one rack-like element in the opposite direction. According to some embodiments, the at least one retaining unit and the at least one rack-like element are physically connected, such that upon rotating the at least one cogwheel, the at least one liquid absorbing element is being moved along the track.

According to some embodiments, conveyer 110 is configured to move mobile liquid absorbing element 108 along the course of track 120.

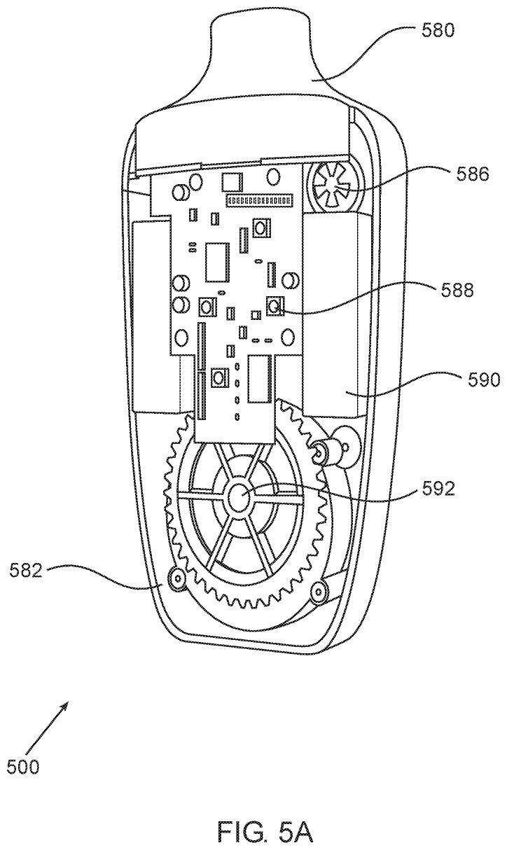

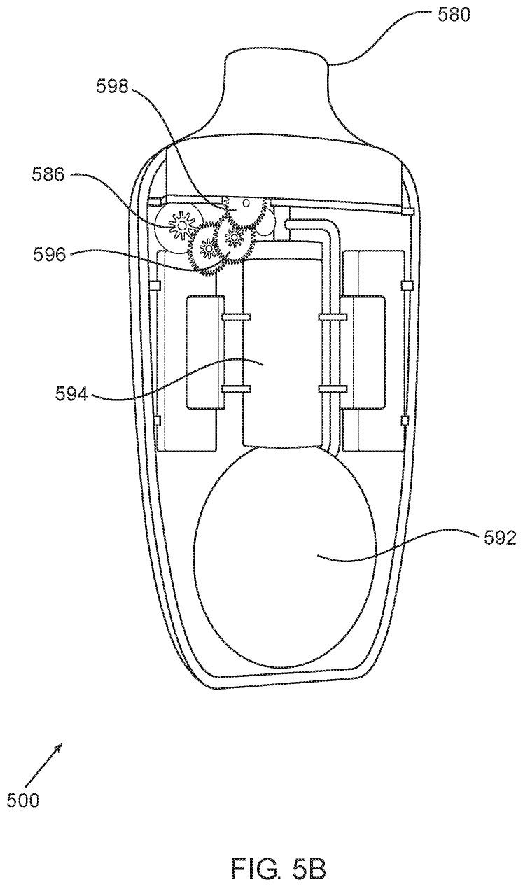

According to some embodiments, the motion along track 120 is enabled by an operating motor, which is located in a control unit, connectable to nebulizer cartridge 100 (not shown) as discussed with reference to FIGS. 5A and 5B.

According to some embodiments, track 120 extends from conveyer 110 to end point 124. Accordingly, and according to some embodiments, the course of track 120 extends from position 134 to end point 124. According to some embodiments, end point 124 is distal from stationary liquid absorbing element 106. According to some embodiments, upon operation of a nebulizer comprising nebulizer cartridge 100 liquid absorbing element 106 travels between position 134 and end point 124, as discussed when referring to FIGS. 5A and 5B. According to some embodiments, upon operation of a nebulizer comprising nebulizer cartridge 100, retaining unit 118 is shifted from position 134 to end point 124, as discussed when referring to FIGS. 5A and 5B.

It is to be understood that when retaining unit 118 together with liquid absorbing element 106 housed therein, travels from position 134 to end point 124, at some point mobile liquid absorbing element 108 does not contact nor face stationary liquid absorbing element 106. Rather, at some point along track 120 mobile liquid absorbing element 108 contacts porous medium 102. As a result, porous medium 102 is wetted by the liquid retained in mobile liquid absorbing element 108. While mobile liquid absorbing element 108 travels along track 120 from position 134 to end point 124 porous medium 102 is covered with a portion of aqueous pharmaceutical composition 116.

According to some embodiments, mobile liquid absorbing element 108 is shifted, by the movement of conveyer 110 on track 120 in parallel to surface 130 of porous medium 102.

According to some embodiments, upon moving along track 120 the at least one mobile liquid absorbing element 108 spreads the first liquid on surface 130 of porous medium 102. According to some embodiments, track 120 is adapted and positioned, such that, when mobile liquid absorbing element 108 travels from position 134 it covers approximately the entire surface of porous medium 102 (not shown).

The terms “spread” and “spreading”, as used herein are to be interpreted broadly and refer to discharge a liquid from one element to another to create a liquid layer which is substantially evenly spread. Preferably, the liquid layer is a thin layer, e.g. having thickness of no more than 1, 0.5, 0.1, 0.05, 0.01 or 0.001 millimeters. Thus, “spreading” includes smearing, covering with, dispersing, laying, daubing, layering, overlaying, wetting, deploying and coating. Spreading of a liquid on a porous medium from a liquid absorbent may be achieved through application of pressure, or by delicate contact between the two elements.

The term “approximately” as used herein may refer to the percentage of surface of the porous medium that may be coated with liquid by the spreading movement of the mobile liquid absorbing element. Approximately may refer to more than 50% coverage, more than 60% coverage, at least 70% coverage, at least 80% coverage, at least 90% coverage or at least 95% coverage. According to some embodiments, porous medium 102 extends along track 120 (not shown). According to some embodiments, porous medium 102 is made of a rigid material. According to some embodiments, porous medium 102 is made of metal. According to some embodiments, porous medium 102 comprises metal. According to some embodiments, porous medium 102 comprises a metal alloy.

According to some embodiments, porous medium 102 has two flat surfaces, one of which is surface 130 which faces mouthpiece and the other is surface 132, which faces pressurized air inlet 112 and/or the control unit (not shown).

The terms ‘medium’ and ‘material’ as used herein with reference to porous elements and materials, are interchangeable.

According to some embodiments, track 120 extends across the surface of at least one porous medium 102.

According to some embodiments, nebulizer cartridge 100 further comprises a pressurized air inlet 112, configured to enable transfer of pressurized air from the pressurized air source in the control unit to nebulizer cartridge 100. According to some embodiments, pressurized air inlet 112 is configured to allow passage of pressurized air from a nebulizer pump to (and through) porous medium 102. According to some embodiments, pressurized air inlet 112 is located proximally to porous medium 102, such that it faces surface 132. Reference to the flow of pressurized air is discussed in greater detail, when referring to FIGS. 5A and 5B.

According to some embodiments, flat surface 130 is facing track 120. According to some embodiments, surface 132 is facing pressurized air inlet 112.

According to some embodiments, porous medium 102 includes a plurality of pores 126.

According to some embodiments, the at least one mobile liquid absorbing element 108 is configured to discharge at least portions of the liquid absorbed therein into, and onto, at least some of the plurality of pores 126.

According to some embodiments, when mobile liquid absorbing element 108 is in position 134 before the first action of nebulizer cartridge 100, porous medium 102 is dry.

According to some embodiments, upon connecting a control unit to cartridge 100, and following its operation (e.g. by pressing a button), mobile liquid absorbing element 108 is moved along track 120 between starting point 122 and end point 124, and spreads aqueous pharmaceutical composition 116 thereon. According to some embodiments, upon connecting a control unit to cartridge 100, and following its operation (e.g. by pressing a button), mobile liquid absorbing element 108 is moved along track 120 between position 134 and end point 124, and spreads aqueous pharmaceutical composition 116 over surface 130. According to some embodiments, during the move, an amount of aqueous pharmaceutical composition 116 is penetrating pores 126 of porous medium 102. According to some embodiments, the penetrating entails wetting porous medium 102.

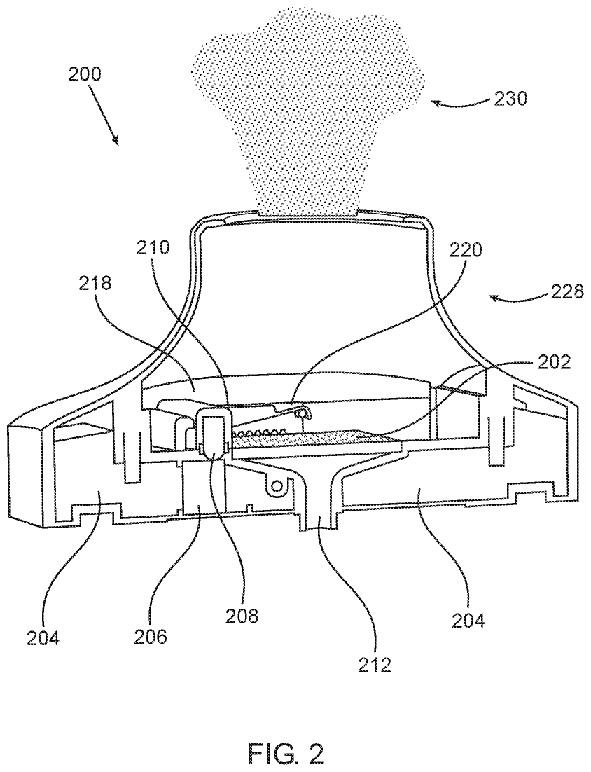

According to some embodiments, upon operation of a nebulizer comprising nebulizer cartridge 100, pressurized air may enter through pressurized air inlet 112 as further detailed with reference to FIGS. 5A and 5B. According to some embodiments, during operation of a nebulizer, the pressurized air entering nebulizer cartridge 100 from pressurized air inlet 112 is hitting surface 132 of porous medium 102, wherein porous medium 102 includes therein a portion of aqueous pharmaceutical composition 116, leading to aerosol formation (as further discussed in reference to FIG. 2). According to some embodiments, the pressurize air is hitting flat surface 132 when porous medium 102 is wet, thereby leading to formation of aerosol. According to some embodiments, the aerosol comprises droplets of aqueous pharmaceutical composition 116. According to some embodiments, the formation of aerosol leaves porous medium 102 substantially dry.

According to some embodiments, the at least one mobile liquid absorbing element is in contact with the at least one stationary liquid absorbing element, for a first time period, which is the time period from the contact and until operating the conveyer. Thus, according to some embodiments, the at least one mobile liquid absorbing element is absorbed with liquid, which is maintained therein until the conveyer is operated, thereafter some of the liquid is discharged onto the porous medium during the traveling of the mobile liquid absorbing element 108 along track 120. As a result, according to some embodiments, at least one porous medium 102 remains dry, or substantially dry, in cartridge 100 until its intended use, i.e. until a user connects the cartridge to the hand held control unit and operates its conveyer motor. According to some embodiments, the at least one porous medium is dried upon application of pressurized air from the control unit, therethrough.

According to some embodiments, snap-fit 114 is located at the edge of nebulizer cartridge 100, such that it faces flat surface 132. According to some embodiments, snap-fit 114 is configured to connect to matching snap-fit, located at the edge of a complementary nebulizer control unit.

Reference is now made to FIG. 2, which schematically illustrates a nebulizer cartridge 200, according to some embodiments. According to some embodiments, nebulizer cartridge 200 is similar to nebulizer cartridge 100. According to some embodiments, nebulizer cartridge 200 includes elements similar to those of nebulizer cartridge 100: at least one porous medium 202 having a plurality of pores; at least one reservoir 204 containing an aqueous pharmaceutical composition; at least one stationary liquid absorbing element 206; at least one mobile liquid absorbing element 208; a conveyer 210 comprising retaining unit 218, and track 220 having starting point and end point; pressurized air inlet 212; and a snap-fit (not indicated in FIG. 2).

According to some embodiments, nebulizer cartridge 200 further includes a mouthpiece 228 configured to enable a user to inhale aerosol 230 formed by a nebulizer having nebulizer cartridge 200.

According to some embodiments, when the pressurized air flows from pressurized air inlet 212 and hits wet porous medium 202, aerosol 230 forms and proceeds through mouthpiece 228 into the respiratory tract of a nebulizer user.

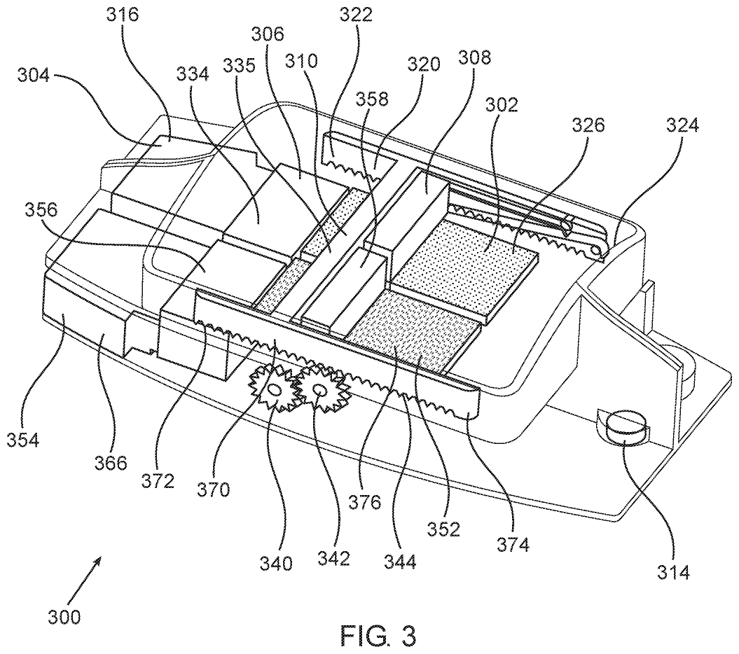

Reference is now made to FIG. 3, which schematically illustrates a nebulizer cartridge 300 comprising a first porous medium 302, a first reservoir 304, a first stationary liquid absorbing element 306, a first mobile liquid absorbing element 308, a conveyer 310, a second porous medium 352 having plurality of pores 326, a second reservoir 354 having plurality of pores 376, a second stationary liquid absorbing element 356, a second mobile liquid absorbing element 358 and a snap-fit 314, according to some embodiments.

According to some embodiments, nebulizer cartridge, e.g. any of nebulizer cartridge 100, 200 or 300 comprises a plurality of porous media.

According to some embodiments, the first stationary liquid absorbing element extends from the first reservoir to the first track, and is in contact with the first liquid contained in the first reservoir, such that upon moving along the first track, the first mobile liquid absorbing element is at least temporarily in contact with the first stationary liquid absorbing element and at least temporarily in contact with the first porous medium.

According to some embodiments, the second stationary liquid absorbing element extends from the second reservoir to the second track; and is in contact with the second liquid contained in the second reservoir, such that upon sliding the second mobile liquid absorbing element on the second track, the second mobile liquid absorbing element is at least temporarily in contact with the second stationary liquid absorbing element and at least temporarily in contact with the second porous medium.

According to some embodiments, each of the first and the second conveyers separately comprises a rack and pinion mechanism.

It is to be understood that the cartridge may include more than two reservoirs, each containing a different liquid. In such cases, as explained with respect to the two-reservoir system, the cartridge may include a respective number of mobile- and stationary liquid absorbing elements and conveyers. Accordingly, the hand held control unit may include the same number of conveyer motors.

It is further to be understood that the inclusion of more than one reservoir (together with matching number of the remaining elements) allows tailor-made nebulizer-based combination therapy. Today, in conventional nebulizer-based combination therapies a number of medications are delivered to the respiratory tract at once. In such cases, both compounds will be delivered to the same region in the respiratory tract depending on the average size of the droplets. However, it may be beneficial to target different active compounds to different locations in the respiratory tract. Duplication of all nebulizer/cartridge element, as portrayed herein, allows to control the droplet sized of each aerosolized composition separately, thus to target different regions in the respiratory tract based on the desired location of each API. According to some embodiments, each of first stationary liquid absorbing element 306, second stationary liquid absorbing element 356, first mobile liquid absorbing element 308 and second mobile liquid absorbing element 358 is individually, a sponge, e.g. a hydrophilic sponge. According to some embodiments, first stationary liquid absorbing element 306, second stationary liquid absorbing element 356, first mobile liquid absorbing element 308 and second mobile liquid absorbing element 358 are made of the same material.

FIG. 3 illustrates a configuration where first mobile liquid absorbing element 308 is not in contact with first stationary liquid absorbing element 306 (as in FIG. 1, with parallel elements), but rather it is in contact with first porous medium 302 as the former is in position 335 (hereinafter, “Configuration B”). Similarly, FIG. 3 illustrates a configuration where the second side of the system is in Configuration B, as second mobile liquid absorbing element 358 is not in contact with second stationary liquid absorbing element 356, but rather it is also in contact with first porous medium 302 as the former is in position 335. According to some embodiments, a configuration, where first mobile liquid absorbing element 308 is in contact with first stationary liquid absorbing element 306 (and second mobile liquid absorbing element 358 is in contact with second stationary liquid absorbing element 356), i.e. Configuration A, precedes Configuration B. Thus, according to some embodiments, in Configuration A, first mobile liquid absorbing element 308 and second mobile liquid absorbing element 358 are contacting first porous medium 302 and second porous medium 352 respectively, when each one of them is wet (i.e. first mobile liquid absorbing element 308 is absorbed with portion of first aqueous pharmaceutical composition 316; and second mobile liquid absorbing element 358 is absorbed with portion of second aqueous pharmaceutical composition 366). According to some embodiments, when first mobile liquid absorbing element 308 and second mobile liquid absorbing element 358 shift from position 334 to position 335, Configuration A shifts to Configuration B. At Configuration B, first mobile liquid absorbing element 308 and second mobile liquid absorbing element 358 transfer portions of first aqueous pharmaceutical composition 316 and second aqueous pharmaceutical composition 366 to first mobile liquid absorbing element 308 and second mobile liquid absorbing element 358, respectively.

According to some embodiments, each of first reservoir 304 and second reservoir 354 acts as a container for holding liquid. According to some embodiments, first reservoir 304 contains first aqueous pharmaceutical composition 316. According to some embodiments, first reservoir 304 is in contact with first stationary liquid absorbing element 306. According to some embodiments, second reservoir 354 contains second aqueous pharmaceutical composition 366. According to some embodiments, second reservoir 354 is in contact with second stationary liquid absorbing element 366.

According to some embodiments, each one of first aqueous pharmaceutical composition 316 and second aqueous pharmaceutical composition 366, separately comprises a therapeutically effective amount of medication for treating one or more medical conditions, which affect the respiratory system

FIG. 3 illustrates Configuration B, where first mobile liquid absorbing element 308 is in contact with first porous medium 302; and second mobile liquid absorbing element 358 is in contact with second porous medium 352, as first mobile liquid absorbing element 308 and second mobile liquid absorbing element 358 are position 335. As a result, when Configuration B is applied, a portion of first aqueous pharmaceutical composition 316 is spread on first porous medium 302 and a portion of second aqueous pharmaceutical composition 366 is spread on second porous medium 352.

As detailed above, when referring to first surface 106a and second surface 106b of stationary liquid absorbing element 106, Configuration A, where a mobile liquid absorbing element is in prolonged contact with a stationary liquid absorbing element leads to an equilibrium, where the absorbing elements and the reservoir, each separately contains a constant amount of liquid, according to some embodiments. According to some embodiments, a transition to position 335, as shown in FIG. 3, leads to a spreading of a portion of first aqueous pharmaceutical composition 316 and second aqueous pharmaceutical composition 366 over first porous medium 302 and second porous medium 352, respectively, in Configuration B. According to some embodiments, the spreading draws out portion of aqueous pharmaceutical composition 316 and second aqueous pharmaceutical composition 366 from first mobile liquid absorbing element 308 and second mobile liquid absorbing element 358, such that upon their return to their original position (i.e. position parallel to position 135), they may absorb further liquids to reach a new equilibrium.

According to some embodiments, conveyer 310 acts in the similar manner to the action of conveyer 110, but while moving two mobile liquid absorbing elements (first mobile liquid absorbing element 308 and second mobile liquid absorbing element 358)

According to some embodiments, conveyer 310 comprises first track 320, second track 370, external cogwheel 340, internal cogwheel 342 and serrated teeth 344.

According to some embodiments, each of first and second mobile liquid absorbing element is movable by the first conveyer on first and second tracks, respectively. According to some embodiments, the conveyer includes a first retaining unit for retaining the first mobile liquid absorbing element and a second retaining unit for retaining the second mobile liquid absorbing element, such that upon moving of the conveyer it forces the movement of the first and second mobile liquid absorbing elements. According to some embodiments, the conveyer comprises a first and a second rack-like elements and corresponding at least one first and at least one second cogwheels. According to some embodiments, the first rack-like element is located along the first track and the second rack-like element is located along the second track. The rest of the mechanism, as detailed with respect to FIG. 1, is duplicated.

According to some embodiments, conveyer 310 is configured to move first mobile liquid absorbing element 308 on the course of first track 320 upon operation from a motor. Conveyer motors and their actions are detailed when referring to FIGS. 5A-B. According to some embodiments, conveyer 310 is also configured to move second mobile liquid absorbing element 358 on the course of second track 320 upon operation of the same motor or other motor.

Preferably, the motor for operating conveyer 310 is not part of nebulizer cartridge 300, but it is rather located in a control unit, which is connectable to of nebulizer cartridge 300. According to some embodiments, the control unit is connectable to of nebulizer cartridge 300, such that an external cogwheel of the motor is interlocked with external cogwheel 340 of conveyer 310, thereby affecting its rotation. According to some embodiments, external cogwheel 340 is interlocked with internal cogwheel 342, which is interlocked with serrated teeth 344 of conveyer 310. As a result, according to some embodiments, conveyer 310 constitutes a “rack and pinion” mechanism, whereby a conveyer motor causes the rotation of external cogwheel 340, internal cogwheel 342 and the motion of first mobile liquid absorbing element 308 and second mobile liquid absorbing element 358 on first track 320 and second track 370 respectively.

All or some of the transitions and manipulations, which take place during the shifting of first mobile liquid absorbing element 308 and second mobile liquid absorbing element 308 over track 320 and track 370 respectively, are similar to those depicted when referring to track 120, starting point 122 (parallel to starting point 322 and starting point 372 in FIG. 3) and end point 124 (parallel to end point 324 and end point 374 in FIG. 3) above.

According to some embodiments, first porous medium 302 and second porous medium 352 are substantially similar to porous medium 102.

It is to be understood that although FIG. 3 depicts a single conveyer, the current disclosure is intended to cover both a single conveyer, which moves first mobile liquid absorbing element 308 and second mobile liquid absorbing element 358; and two separate independent conveyers, wherein a first conveyer moves first mobile liquid absorbing element 308 and a second conveyer moves second mobile liquid absorbing element 358. In such cases, according to some embodiments, the wetting of first porous medium 302 and second porous medium 352 with first aqueous pharmaceutical composition 316 and second aqueous pharmaceutical composition 366, respectively, may be simultaneous or consecutive. According to some embodiments, the wetting of the two may be in the same rate/frequency or at different rates. According to some embodiments, the wetting may forms similar amounts (e.g. similar volumes, masses, or concentrations of active material) of pharmaceutical compositions in each medium or different amounts. Basically, a two-reservoir system as cartridge 300 depicted is FIG. 3, it intended to deliver two (optionally, different) pharmaceutical compositions. Thus, according to some embodiments first pharmaceutical composition 316 and second aqueous pharmaceutical composition 366 do not consist of the same pharmaceutically active ingredients. According to some embodiments, a nebulizer comprising nebulizer cartridge 300 may deliver the two distinct pharmaceutical compositions at once to form a single aerosol, or in two aerosolization cycles to deliver each composition to its intended location in the respiratory tract. For example, control of the amount of a pharmaceutical composition in the porous medium and its formulation may result in a control of the diameter of aerosol droplets. Thus, according to some embodiments, different specifications to two separate pharmaceutical compositions may lead to different droplet-sized aerosols, which reach different locations in the lungs, where both aerosols are stemming from the same nebulizer.

The correlation between droplet size and deposition thereof in the respiratory tract has been established. Droplets around 10 micron in diameter are suitable for deposition in the oropharynx and the nasal area; droplets around 2-4 micron in diameter are suitable for deposition in the central airways (and may be useful for delivering a bronchodilator, such as, salbutamol) and droplets smaller than 1 micron in diameter are suitable for delivery to the alveoli (and may be useful for delivering pharmaceuticals to the systemic circulation, for example, insulin).

Advantageously, the devices, systems and methods disclosed herein provide a relatively uniform or homogeneous wetting of the porous surface that may result in spreading a small diameter aerosol droplets, and confer the ability to yield such small diameter aerosol drops with high efficiency.

According to some embodiments, the at least one mobile liquid absorbing element (e.g. at least one mobile liquid absorbing element 108) is configured to homogeneously or semi-homogeneously spread the liquid across the surface of the at least one porous medium (e.g. at least one porous medium 102) upon moving on the track (e.g. track 12). According to some embodiments, the spreading is homogeneous.

The terms ‘droplet size’ and ‘mass median aerodynamic diameter’, also known as MMAD, as used herein are interchangeable. MMAD is commonly considered as the median particle diameter by mass.

According to some embodiments, droplets of the aerosol produced by the method and nebulizers disclosed herein are having an MMAD within the range of 0.3 to 7 microns. According to some embodiments, the MMAD is within the range of 2 to 10 microns. According to some embodiments, the MMAD is less than 5 microns.

According to some embodiments, control over droplet size and modality of generated aerosol is achieved by controlling physical properties of the porous medium. According to some embodiments, the physical properties of the porous medium are adjusted based on the desired droplet size. The physical properties of the porous medium, may include, but are not limited to, physical dimensions of the porous medium as a whole, pore count, pore density, pore distribution, pore shape, homogeneity of the aforementioned pore features, hydrophobicity of the porous material, and electromagnetic affinity among other properties. Each possibility is a separate embodiment of the invention.

The term “modality” as used herein refers to the modality of size distributions and includes, but is not limited to, uni-modal, bi-modal and tri-modal size distributions.

According to some embodiments, control over droplet size and modality of generated aerosol is achieved by controlling the properties of the medication and/or liquid and/or composition. The properties of the medication and/or liquid and/or composition which may be adjusted to achieve the desired aerosol, include, but are not limited to, viscosity, surface tension, pH, electrolyte concentration, solid content and polarity

According to some embodiments, Snap-fit 314 is located at the edge of nebulizer cartridge 300 and is configured to connect to another (matching) snap-fit mechanism, located at the edge of a complementary nebulizer control unit.

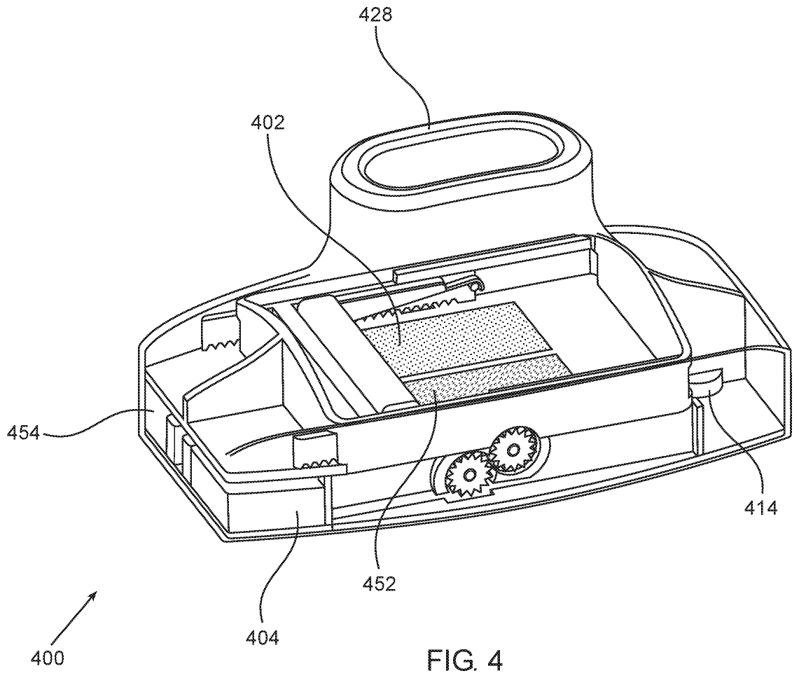

Reference is now made to FIG. 4, which schematically illustrates a nebulizer cartridge 400, according to some embodiments. According to some embodiments, nebulizer cartridge 400 is similar to nebulizer cartridge 300. According to some embodiments, nebulizer cartridge 400 includes elements similar to those of nebulizer cartridge 300: a first porous medium 402, a second porous medium 452, a first reservoir 404, a first stationary liquid absorbing element (not shown), a first mobile liquid absorbing element (not shown), a conveyer, a second reservoir 454, a second stationary liquid absorbing element (not shown), a second mobile liquid absorbing element (not shown) and a snap-fit 414.

According to some embodiments, nebulizer cartridge 400 further comprises a mouthpiece 428 for enabling a user to inhale an aerosol(s) formed by a nebulizer having nebulizer cartridge 400. Specifically, when pressurized air flows from a pressurized air source in a nebulizer control unit and hits wet first porous medium 402 and/or second porous medium 452, the aerosol(s) form and proceed through mouthpiece 428 into the respiratory tract of a nebulizer user, according to some embodiments.

Reference is now made to FIGS. 5A and 5B, each schematically illustrate a perspective sectional view of nebulizer 500, according to some embodiments. Nebulizer 500 comprises a nebulizer cartridge 580, which may be similar to any one of nebulizer cartridges 100, 200, 300 or 400; and a nebulizer control unit 582.

According to some embodiments, the control unit comprises a conveyer motor having a gear unit and a pressurized air source; wherein said nebulizer cartridge is configured to be mounted on the control unit, such that upon mounting, at least one cogwheel of the gear operates the at least one conveyer.

According to some embodiments, the least one conveyer comprises a rack and pinion mechanism. According to some embodiments, the nebulizer cartridge is configured to be mounted on the control unit, such that upon mounting, at least one cogwheel of the gear operates the rack and pinion mechanism, and the at least one conveyer is actuated by the conveyer motor.

According to some embodiments, the control unit is a hand held control unit.

According to some embodiments, the nebulizer is mobile. According to some embodiments, the nebulizer is handheld. According to some embodiments, the nebulizer is powered by a mobile power source.

It is to be understood that the nebulizer disclosed herein is a two-part configuration, i.e. the cartridge and the hand held control unit are separate units, according to some embodiments. This structural configuration allows easy maintenance due to separation between the constant, reusable part and disposable parts. It is also cost effective, since the control unit is commonly more expensive than the disposable unit. In most nebulizers, all elements, but the reservoir of pharmaceutical composition are marketed for long periods of use, while the reservoir of pharmaceutical composition are disposable as they can be used for single or very few applications. This configuration, however, requires frequent cleaning and maintenance of rather inexpensive nebulizer parts, in which cannot be separated from the non-disposable nebulizer unit (such as, the mouthpiece). This is while the more expensive, non-disposable, parts of the nebulizer unit (e.g. motors and pumps) do not require frequent maintenance. The configuration disclosed herein separates the durable, non-disposable, relatively more expensive from the disposable, inexpensive elements to two units. The control unit is intended for long periods use (until it is worn out), while the reservoir, aerosolization liquid, sponges and porous media are disposables. This way, the expensive elements do not require almost any maintenance and can survive numerous replacements of the disposable units, during numerous applications of the nebulizer.

According to some embodiments, the nebulizer cartridge and the control unit are interconnectable. According to some embodiments, the nebulizer cartridge comprises a first attachment element and the control unit comprises a second attachment element. According to some embodiments, the first and second attachment elements are interconnectable. According to some embodiments, the first attachment element comprises a first snap-fit. According to some embodiments, the second attachment element comprises a second snap-fit. According to some embodiments, the first and second snap-fits are interconnectable.

According to some embodiments, the first attachment element is a magnet and the second attachment element is a piece capable of being attached to a magnet. According to some embodiments, the first attached element is a mounting means and the second attachment element is a groove adapted to attach to the unit comprising the first attachment element, through the first attachment element.

According to some embodiments, nebulizer cartridge 580, and nebulizer control unit 582 may be provided as separate units. Preferably, nebulizer cartridge 580, and nebulizer control unit 582 are interconnectable.

According to some embodiments, nebulizer control unit 582 is a hand held unit, which is operated by a nebulizer user in need for inhaling an aerosolized pharmaceutical composition. According to some embodiments, nebulizer control unit 582 comprises a conveyer motor 586, a computing unit 588, electric power source 590 and pressurized air source 592.

According to some embodiments, conveyer motor 586 is located in nebulizer control unit 582. According to some embodiments, conveyer motor 586 is powered by electric power source 590. According to some embodiments, conveyer motor 586 is operated by computing unit 588. According to some embodiments, conveyer motor 586 comprises a set of conveyer motor of cogwheels 596.

According to some embodiments, upon mounting of the nebulizer cartridge on the control unit, at least one cogwheel of the gear operates the rack and pinion mechanism of the first conveyer, and the first conveyer is actuated by the conveyer motor; and at least one cogwheel of the gear operates the rack and pinion mechanism of the second conveyer, and the second first conveyer is actuated by the conveyer motor.

According to some embodiments, the conveyer motor is configured to rotate at least one conveyer motor cogwheel. According to some embodiments, the at least one conveyer motor cogwheel and the conveyer motor are located in the control unit. According to some embodiments, each one of at least one conveyer motor cogwheel includes serrated teeth. According to some embodiments, the at least one conveyer motor cogwheel comprises an external conveyer motor cogwheel comprising serrated teeth. According to some embodiments, rotating the at least one conveyer motor cogwheel by the motor entails rotating the serrated teeth of the external conveyer motor cogwheel. According to some embodiments, the control unit and the nebulizer cartridge are interconnectable, such that upon their connection, the serrated teeth of the external conveyer motor cogwheel are interlocked with the serrated teeth of the external cogwheel of the at least one rack-like element of the conveyer. According to some embodiments, the interlocking entails that upon rotating said external conveyer motor cogwheel, its teeth are rotating a radial direction, and pushing the interlocked teeth of the external cogwheel of the at least one rack-like element, such that the external cogwheel of the at least one rack-like element are rotated in the same direction. As detailed above, the rotation of the cogwheel of the at least one rack-like element causes the movement of the at least one rack-like element at a tangential direction in a rack and pinion mechanism, according to some embodiments. According to some embodiments, rotating said external conveyer motor cogwheel in the opposite direction entails inversion of the process, thus moving the at least one rack-like element in the opposite direction. As a result, the operation of the conveyer motor entails the movement or sliding of the at least one mobile liquid absorbing element along the track.

According to some embodiments, the conveyer motor is configured to be actuated by a user.

According to some embodiments, set of conveyer motor of cogwheels 596 includes an external conveyer motor cogwheel 598. According to some embodiments, external conveyer motor cogwheel 598 is rotating together with set of conveyer motor of cogwheels 596 by conveyer motor 586 as a result from instruction(s) from computing unit 588. According to some embodiments, external conveyer motor cogwheel 598 is interlocking with an external cogwheel of the conveyer of nebulizer cartridge 580, such that upon rotation of external conveyer motor cogwheel 598, a rack and pinion mechanism operates to affect the movement of a mobile sponge(s) as detailed above with reference to FIG. 3.

According to some embodiments, pressurized air source 592 is located in nebulizer control unit 582. According to some embodiments, pressurized air source 592 is an air pump, configured to produce pressurized gas. Specifically, pressurized air source 592 is configured to produce pressurized air from atmospheric air, according to some embodiments. Pressurized air source 592 comprises air pump motor 594, which is powered by electric power source 590 and operated by computing unit 588, according to some embodiments. According to some embodiments, air pump motor 594 affects the formation of pressurized air in pressurized air source 592.

According to some embodiments, the pressurized air source is configured to deliver pressurized gas through the pressurized air inlet to the porous medium and create an ultra-atmospheric pressure on one side of the porous medium, thereby induce a pressure gradient at the porous medium. According to some embodiments, the pressurized air source is configured to deliver pressurized gas through the pressurized air inlet to the porous medium and create an ultra-atmospheric pressure the second side of the porous medium, thereby induce a pressure gradient at the porous medium.

The term ‘pressurized air’ as used herein is interchangeable with the term ‘compressed air’ and refers to air under pressure above atmospheric pressure.

According to some embodiments, the control unit comprises a pump motor, configured to operate the pump.

According to some embodiments, the computing unit is configured to operate the pump motor.

According to some embodiments, computing unit 588 is located in nebulizer control unit 582. According to some embodiments, computing unit 588 is powered by electric power source 590. According to some embodiments, computing unit 588 is operated by a nebulizer user. according to some embodiments, upon receiving an instruction(s) from the nebulizer user, computing unit 588 instructs conveyer motor to affect the rotation of set of conveyer motor of cogwheels 596, which eventually, as described above results in the movement of a wet mobile sponge(s) towards a porous medium or media. As detailed herein, the process is resulting in the wetting of the porous medium/media.

According to some embodiments, upon receiving an instruction(s) from the nebulizer user, computing unit 588 instructs air pump motor 594 to affect to operation of pressurized air source 592 and thereby create pressurized air. The formed pressurized air exist nebulizer control unit 582 and enters nebulizer cartridge 580, through an air inlet located in nebulizer cartridge 580 in proximity to its connection surface with nebulizer control unit 582, according to some embodiments. According to some embodiments, after entering nebulizer control unit 582 pressure difference therein results in the pressurized air proceeding towards and hitting the porous medium/media thereby forming an aerosol, upon instruction of the nebulizer user.

According to some embodiments, the control unit comprises a computing unit configured to operate the conveyer motor. According to some embodiments, the computing unit is controlled by a user.

According to some embodiments, the control unit comprises a computing unit configured to operate each of conveyer motors. According to some embodiments, the control unit comprises a computing unit configured to operate the first conveyer motor. According to some embodiments, the control unit comprises a computing unit configured to operate the second conveyer motor.

According to some embodiments, electric power source 590 is located in nebulizer control unit 582 and may include rechargeable batteries, where it is configured to power air pump motor 594 and computing unit 588.

According to some embodiments, nebulizer cartridge 580 has a similar configuration to that of any one nebulizer cartridge 100, nebulizer cartridge 200, nebulizer cartridge 300, or nebulizer cartridge 400. According to some embodiments, nebulizer cartridge 580 includes elements similar to those of the above nebulizer cartridges: one or more porous media, one or more reservoirs, one or more stationary sponges, one or more mobile sponges, one or more conveyers and a snap-fit.

According to some embodiments, there is provided a method for producing aerosols, the method comprises:

providing the nebulizer disclosed herein;

obtaining instructions from a user to operate the conveyer motor(s);

operating the conveyer motor(s) thereby spreading the liquid onto the surface of the at least one porous medium; and

operating the pressurized air source thereby introducing pressure gradient to the at least one porous medium and thereby producing aerosol, wherein the aerosol comprises droplets of the liquid.

According to some embodiments, the method comprises connecting the control unit and the nebulizer cartridge, such that upon their connection, the serrated teeth of the external conveyer motor cogwheel are interlocked with the serrated teeth of the external cogwheel of the at least one rack-like element of the conveyer.

According to some embodiments, obtaining instructions from a user comprises obtaining instructions to the computing unit. According to some embodiments, upon receiving instructions in the computing unit, the computing unit sends a signal to the conveyer motor to turn on and operate. According to some embodiments, upon operation of the conveyer motor, it rotates the at least one conveyer motor cogwheel. According to some embodiments, rotating the at least one conveyer motor cogwheel by the motor entails rotating the serrated teeth of the external conveyer motor cogwheel. According to some embodiments, upon rotating said external conveyer motor cogwheel, its teeth are rotating in a radial direction, and pushing the interlocked teeth of the external cogwheel of the at least one rack-like element, such that the external cogwheel of the at least one rack-like element are rotated in the same direction.

According to some embodiments, the serrated teeth of the at least one rack-like element are interlocking with the serrated teeth of the external cogwheel, such that upon rotating said interlocking external cogwheel, its teeth are rotating a radial direction, and pushing the interlocked teeth of the at least one rack-like element, such that the at least one rack-like element is moved at a tangential direction in a rack and pinion mechanism. According to some embodiments, rotating said external cogwheel in the opposite direction entails moving the at least one rack-like element in the opposite direction. According to some embodiments, the at least one retaining unit and the at least one rack-like element are physically connected, such that upon rotating the at least one cogwheel, the at least one liquid absorbing element is being moved along the track.

According to some embodiments, upon receiving instructions the computing unit sends a signal to the conveyer motor to rotate the at least one conveyer motor cogwheel in the opposite direction, thereby inverting of the process and moving the at least one rack-like element in the opposite direction. As a result, obtaining instructions from a user entails affecting the movement or sliding of the at least one mobile liquid absorbing element along the track at any desired direction.

According to some embodiments, upon receiving instructions in the computing unit, the computing unit sends a signal to the pressurized air source to turn on and operate. According to some embodiments, the pressurized air source is an air pump having an air pump motor. According to some embodiments, upon receiving instructions, the computing unit sends a signal to the air pump motor to turn on and operate.

According to some embodiments, the air pump comprises blades. According to some embodiments, upon operation of the air pump motor, the air pump motor rotates the blades. According to some embodiments, the rotating of the blades creates pressurized air (i.e. positive air pressure). According to some embodiments, the pressurized air exits the control unit and enters the nebulizer cartridge, through the air inlet. According to some embodiments, the entering of the pressurized air to the nebulizer cartridge results in the pressurized air hitting the at least one porous medium, thereby creating aerosol. According to some embodiments, the aerosol exits the nebulizer cartridge through the mouthpiece. As a result, operating the pressurized air source and conveyer motor through instructions from the user to the computing unit, results in the wetting of the at least one porous medium and hitting it with pressurized air, such that the wetting liquid is aerosolized and the aerosol exits the nebulizer through the mouthpiece.

According to some embodiments, operating the conveyer motor(s) comprises instructing the computing unit to operate the conveyer motor. According to some embodiments, the instructing is performed by a user. According to some embodiments, instructing the computing unit entails determining a desired amount of aerosol to be produced; wherein operating the conveyer motor(s) is repeated for a number of times in response to the desired amount of aerosol.

According to some embodiments, the method further comprises delivering the aerosols to the respiratory system of a subject in need thereof.

The nebulizer disclosed herein may function as an inhaler under some circumstances. Thus, the terms ‘nebulizer’ and ‘inhaler’ as used herein may be interchangeable.

According to some embodiments, the nebulizer is configured to communicate wirelessly with servers, databases, personal devices (computers, mobile phones) among others.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1. A nebulizer cartridge comprising
at least one porous medium having an proximal surface, the at least one porous medium extending between a first position and a second position;
at least one reservoir configured to contain a liquid;
at least one mobile liquid absorbing element;
at least one stationary liquid absorbing element being in contact with the at least one reservoir;
at least one conveyer connected to the at least one mobile liquid absorbing element, and configured to be actuated by a motor; and
a track operably linked to the at least one conveyer,
wherein said at least one conveyer and said at least one mobile liquid absorbing element connected thereto are configured to move along the track; and wherein the at least one mobile liquid absorbing element is configured to be in contact with the at least one stationary liquid absorbing element, and upon movement along the track it is further configured to be in contact with the at least one porous medium.

2. The nebulizer cartridge of claim 1, wherein the at least one reservoir contains the liquid, and wherein said at least one stationary liquid absorbing element is in contact with the liquid contained in the reservoir.

3. The nebulizer cartridge of claim 2, wherein each of the at least one stationary liquid absorbing element and the at least one mobile liquid absorbing element, separately, is configured to absorb liquid in an amount which is at least 150% of its respective weight.

4. The nebulizer cartridge of claim 3, wherein each of the at least one stationary liquid absorbing element and the at least one mobile liquid absorbing element comprises cloth, wool, felt, sponge, foam, cellulose, yarn, microfiber or a combination thereof.

5. The nebulizer cartridge of claim 2, wherein the track extends along the first position and the second position of the at least one porous medium.

6. The nebulizer cartridge of claim 1, further comprising a mouthpiece, such that the proximal surface of the at least one porous medium is facing the mouthpiece.

7. The nebulizer cartridge of claim 1, wherein the at least one porous medium is having an distal surface, opposing the proximal surface, and nebulizer cartridge further comprises a pressurized air inlet, such that the distal surface of the at least one porous medium is facing the pressurized air inlet.

8. The nebulizer cartridge of claim 2, wherein the at least one stationary liquid absorbing element contains a portion of the liquid, absorbed therein, and wherein the at least one mobile liquid absorbing element is in contact with the at least one stationary liquid absorbing element, thereby absorbing liquid therefrom.

9. The nebulizer of claim 8, wherein the at least one mobile liquid absorbing element is in contact with the at least one porous medium, thereby wetting the proximal surface thereof.

10. The nebulizer of claim 9, wherein said wetting comprises spreading.

11. The nebulizer cartridge of claim 2, wherein the liquid comprises an aqueous solution or an aqueous suspension of a pharmaceutical composition.

12. The nebulizer cartridge of claim 2, comprising a plurality of porous media; a plurality of reservoirs, each containing a liquid; a plurality of mobile liquid absorbing elements; a plurality of stationary liquid absorbing elements; and a plurality of conveyers, each configured to be actuated by a respective motor, wherein each of said plurality of reservoirs contains a different liquid.

13. (canceled)

14. A nebulizer comprising:
the nebulizer cartridge of claim 1, wherein the least one conveyer comprises a rack and pinion mechanism; and
a control unit;
wherein said control unit comprises a conveyer motor having a gear unit and a pressurized air source; wherein said nebulizer cartridge is configured to be mounted on the control unit, such that upon mounting, at least one cogwheel of the gear operates the rack and pinion mechanism, and the at least one conveyer is actuated by the conveyer motor.

15. (canceled)

16. The nebulizer of claim 14, wherein the control unit comprises a computing unit configured to operate the conveyer motor.

17. The nebulizer of claim 16, wherein the computing unit is controlled by a user.

18. The nebulizer of claim 16, wherein the pressurized air source comprises an air pump.

19. The nebulizer of claim 18, wherein the control unit comprises a pump motor, configured to operate the air pump.

20. The nebulizer of claim 19, wherein the control unit comprises an electric power source, configured to power the computing unit, the pump motor and the conveyer motor.

21. The nebulizer of claim 14, wherein the nebulizer cartridge further comprises a pressurized air inlet, configured to enable transfer of pressurized air from the pressurized air source to the nebulizer cartridge.

22. The nebulizer of claim 21, wherein the nebulizer cartridge further comprises a mouthpiece, such that upon application of the pressurized air source, pressurized air flows therefrom, through the at least one porous medium, thereby producing aerosol, which flows out the nebulizer cartridge through the mouthpiece.
